(19) United States
(12) Patent Application Publication
(10) Pub. No.: US 2020/0030349 A1
(43) Pub. Date: Jan. 30, 2020

(54) PHYSIOLOGICALLY ACTIVE PREPARATION COMPRISING N-ACETYL-GLUCOSAMINE FOR THE TREATMENT OF BACK PAIN

(71) Applicant: Natural Products & Drugs GmbH, Spittal/Drau (AT)

(72) Inventor: Josef Schantl, Seeboden (AT)

(73) Assignee: Natural Products & Drugs GmbH, Spittal/Drau (AT)

(21) Appl. No.: 16/498,811

(22) PCT Filed: Mar. 28, 2018
(86) PCT No.: PCT/EP2018/057910

§ 371 Date: Sep. 27, 2019

(30) Foreign Application Priority Data
Mar. 28, 2017 (EP) 17163233.4

(51) Int. Cl. A61K 31/7008; A61K 36/899; A61K 31/198; A61K 47/36; A61K 47/22; A61K 47/18; A61K 47/02; A61K 47/42; A23L 33/105; A23L 33/18; A23L 29/281; A23L 29/00; A61P 29/00

(52) U.S. Cl. CPC A61K 31/7008; A61K 36/899; A61K 31/198; A61K 47/36; A61K 47/22; A61K 47/183; A61K 9/0053; A61K 47/42; A23L 33/105; A23L 33/18; A23L 29/284; A23L 29/015; A61P 29/00; A61K 47/02

(57) ABSTRACT

A physiologically active composition for use in a method for the prophylaxis and/or treatment of back pain comprises N-acetyl-glucosamine or a suitable derivative of N-acetyl-glucosamine.

### TECHNICAL FIELD

The invention relates to a physiologically active composition for use in a method for the prophylaxis and/or treatment of back pain. The invention relates further to a physiologically active composition which comprises at least two components, of which a first component is silica.

### BACKGROUND

Ever more people are being affected at an ever younger age by back pain at rest and/or on exertion. Causes of such back pain, in addition to age-related degeneration, are too little movement, excessive loading and/or incorrect loading. For example, sitting for hours on end in an office job can lead to severe back pain.

Such back pain is generally due to degenerative, or wear-related, changes in the vertebral column, which lead to vertebral column pain. The vertebral column comprises a plurality of vertebrae each composed of a vertebral body and a vertebral arch, as well as an intervertebral disc situated between two vertebrae. The vertebrae and vertebral arches are connected together and stabilised by associated vertebral column ligaments.

The intervertebral discs already begin to wear when a person reaches the age of about 20, when the person stops growing. The intervertebral discs gradually lose their cushioning function between the vertebrae. Increasing wear of and change in the vertebrae as well as muscle stiffness as a result of an accompanying change in the vertebral column statics are the result. There is also a risk of intervertebral disc prolapse. Associated therewith is pain at rest and/or on exertion, which at least sometimes is persistent. Wear of the intervertebral discs is the cause of about 70 percent of all back pain.

In the search for relief, affected people often turn to over-the-counter or prescription medicines, undergo lengthy physiotherapy treatments or even surgical interventions.

### SUMMARY

The problem on which the invention is based is to provide a composition which effectively serves for the prophylaxis and/or treatment of back pain. Degenerative vertebral column pain in particular is to be prevented or alleviated thereby.

This problem is solved according to the invention by a physiologically active composition comprising N-acetyl-glucosamine or a derivative of N-acetyl-glucosamine for use in a method for the prophylaxis and/or treatment of back pain.

N-Acetyl-glucosamine (NAG), or N-acetyl-D-glucosamine, is the amide of glucosamine and ethanoic acid or acetic acid. Other names for N-acetyl-glucosamine are 2-acetamido-2-deoxy-D-glucopyranose or 2-acetamido-2-deoxy-β-glucopyranose or 2-(acetylamino) -2-deoxy-D-glucose. A derivative of N-acetyl-glucosamine is to be understood as being a derivative in which N-acetyl-glucosamine is present as the basic structure. In particular, a different atom or a different atom group is provided in place of an H atom of N-acetyl-glucosamine. In the present description, for reasons of better readability, the term N-acetyl-glucosamine always includes such a derivative.

By means of the composition according to the invention comprising N-acetyl-glucosamine, a surprisingly good effect in the prophylaxis and/or treatment of back pain can be achieved.

Because of the binding of its nitrogen in the ethanoic acid amide, N-acetyl-glucosamine is particularly stable towards a reaction with other substances. In addition, it has been found according to the invention that N-acetyl-glucosamine is bioavailable in the body particularly quickly and in a large amount.

A comparison of N-acetyl-glucosamine with glucosamine shows that glucosamine does not occur in free form. Glucosamine is a conventional nutritional supplement and medicament which is used to promote joint health and in arthrosis of the knee joint. Glucosamine is thereby always in the form of glucosamine sulfate or glucosamine hydrochloride, which is additionally stabilised by the addition of potassium chloride or sodium chloride. Otherwise, glucosamine would react with itself and with other substances because of free valences of its nitrogen.

Furthermore, a more rapid and higher bioavailability of N-acetyl-glucosamine has been found in particular in comparison with glucosamine sulfate used orally. The bioavailability of N-acetyl-glucosamine is more than three times as high as the bioavailability of glucosamine sulfate, measured on the basis of how much of the substance in question is actually available in the tissue following oral ingestion.

Glucosamine can be converted to N-acetyl-glucosamine in the body, but such a conversion takes time. In addition, some orally ingested glucosamine, which is burned by the body as an energy source or directly excreted again, is lost.

With the composition according to the invention comprising N-acetyl-glucosamine, the human body can be provided with a direct precursor of hyaluronic acid and other glycosaminoglycans (GAG) or mucopolysaccharides. Production of hyaluronic acid and other glycosaminoglycans in the body can thus effectively be increased. As a glycosaminoglycan, hyaluronic acid has a macromolecular chain of a repeating disaccharide unit, which is formed of D-glucuronic acid and N-acetyl-D-glucosamine. Hyaluronic acid occurs in various types of tissue and fulfils many functions. The storage of water in the various tissues is especially a prominent function of hyaluronic acid.

Particularly positive effects of promoting hyaluronic acid production in the body in this way by supplying N-acetyl-glucosamine have been found on the intervertebral discs.

The intervertebral disc is a connection between two vertebrae of fibrous cartilage tissue or fibrocartilage. Such a connection is a cartilaginous bone connection or symphysis. A symphysis is not a true joint and differs fundamentally from a fibrocartilaginous articular disc in a true joint, such as in a knee joint, shoulder joint, hip joint, etc. In a true joint there is a joint space between two bone ends and the bone ends are covered by a layer of cartilage. Between the layers of cartilage is the articular disc, which is composed of fibrocartilage and tight connective tissue of parallel fibres.

By contrast, the intervertebral disc comprises an outer fibrous ring and an inner gelatinous core. The fibrous ring has on the outside concentric layers of collagenous connective tissue fibres which inwardly merge into fibrocartilage. The connective tissue fibres adhere to the vertebrae and thus connect the vertebrae together. Inside the fibrous ring is the gelatinous core, which is a jelly-like tissue. The tissue contains 80 to 85 percent water and a matrix of hyaluronic acid and proteoglycans, the side chains of which are glycosaminoglycans. The tissue is thus able to reversibly bind comparatively large amounts of water and act in a shock-absorbing manner as a type of water cushion.

In the course of a lifetime, the intervertebral discs wear noticeably. The gelatinous core thereby loses its matrix of hyaluronic acid and proteoglycans. The gelatinous core increasingly dries out, so that its volume decreases and the vertebral bodies move closer to one another. In addition, the cushioning function of the intervertebral discs is lost. The vertebral bodies are excessively loaded and worn, which leads to back pain.

When N-acetyl-glucosamine was supplied, it was possible to observe that back pain due in particular to degenerative changes in the intervertebral discs can reliably be reduced and eliminated. N-Acetyl-glucosamine effectively increases the formation of hyaluronic acid and proteoglycans in the gelatinous core and improves the water storage function of the gelatinous core. In addition, N-acetyl-glucosamine has an anti-inflammatory and pain-relieving effect there. The intervertebral discs can thus be particularly effectively maintained and/or built up by means of N-acetyl-glucosamine. A distance between the vertebral bodies can be increased again. Such causes of back pain can be treated correspondingly effectively. In an ideal case, back pain can even be prevented.

Advantageously according to the invention, the composition additionally comprises silica. Within the meaning of the present invention, the term silica includes silica having the general chemical formula H₂ₙ₊₂SiₙO₃ₙ₊₁, amorphous silica, amorphous or non-crystalline silicon dioxide, natural or organic silica and natural silicon dioxide. Silica is thus overall a supplier of silicon, which is an essential trace element for humans. Silicon is required in the body for the production of keratin in the hair and nails, for wound healing and for regeneration of connective tissue. To that end, silicon has a stimulating effect on the synthesis of glycosaminoglycans and collagen, a fibrous structural protein of the connective tissue. Silicon thus contributes to the elasticity of the connective tissue and ensures the stability thereof.

In the advantageous combination according to the invention of silica with N-acetyl-glucosamine, a particularly effective composition for use in a method for the prophylaxis and/or treatment of back pain is provided. The silica enhances the action of N-acetyl-glucosamine in that crosslinking of the glycosaminoglycans, of collagen and elastin, a further fibrous structural protein, is promoted. An interaction of the body structures so crosslinked cushions impacts that occur particularly strongly. Forces occurring at the intervertebral discs and vertebrae can thus be dissipated particularly quickly and extensively into the ligaments of the vertebral column and further elastic structures of the vertebral column.

In addition, silica has the effect that the fibrous ring of the intervertebral disc remains elastic for its function. The fibrous ring comprises connective tissue, which can be stabilised by means of the silicon of the silica. In addition, N-acetyl-glucosamine has positive effects on the water storage function of the connective tissue by stimulating hyaluronic acid formation in the connective tissue. The fibrous ring can thus be prevented from drying out and becoming brittle. A tear in the fibrous ring would otherwise lead to intervertebral disc prolapse. In the case of intervertebral disc prolapse, the gelatinous core escapes into a vertebral canal formed by vertebra located one above the other. In the vertebral canal is the spinal cord, on which the gelatinous core then presses and causes pain.

According to the invention, the silica is particularly advantageously provided in the form of a constituent of a bamboo extract. Natural silicon dioxide, or natural silica, is present in abundance in the bamboo extract.

The bamboo extract is conventionally obtained from bamboo, in particular from a specific species of bamboo, namely from Bambusa arundinacea and Bambusa vulgaris. A secretion from cavities of the bamboo, so-called bamboo camphor or tabashir, is used for this purpose. Tabashir is a silicon-rich substance which contains essentially amorphous silica and serves as the basis for the bamboo extract. The bamboo extract thus comprises essentially natural silicon dioxide.

Advantageously according to the invention, the bamboo extract contains natural silicon dioxide in an amount by mass, or amount by weight, of from 60 to 90 percent by mass, or percent by weight, preferably from 70 to 80 percent by weight and particularly preferably 75 percent by weight, based on the bamboo extract. An amount of silicon dioxide that is sufficiently high and has a correspondingly good activity is thus contained in the bamboo extract.

Advantageously according to the invention, the bamboo extract further has a mass ratio relative to N-acetyl-glucosamine of from 0.5 to 0.9, preferably from 0.6 to 0.8 and particularly preferably of 0.7. With such a mass ratio, a particularly good interaction could be achieved in particular in the treatment of back pain.

Furthermore, the composition according to the invention advantageously additionally comprises collagen. A supply of collagen has a positive effect on the health of joints and can prevent osteoporosis. Collagen is a fibrous structural protein of the connective tissue and is the most widespread protein in the human body. As a structure-giving protein, collagen is the most important building substance of the vertebral bodies and vertebral arches as well as of all cartilage of the intervertebral discs. Collagen thus plays a substantial part in building the vertebral column. Furthermore, collagen strengthens tendons and skeletal muscles as well as the ligaments of the vertebral column. The supply of collagen in combination with N-acetyl-glucosamine and/or silica synergistically enhances the positive effect thereof on the health of the vertebral column.

Particularly advantageously according to the invention, collagen is provided in the form of a constituent, or component, of a collagen hydrolysate. The collagen hydrolysate comprises hydrolysed collagen. Collagen is thereby enzymatically or chemically solubilised and can thus be more easily digested and taken up by the body. The positive effect of collagen can better be utilised and accelerated. In addition, formation of type II collagen, which functions as a structural protein of hyaline and elastic collagen, in the cartilage-forming cells is stimulated by means of collagen hydrolysate.

The collagen hydrolysate preferably comprises collagen in an amount by mass of from 50 to 80 percent by weight and particularly preferably from 60 to 70 percent by weight. A high and correspondingly active amount of collagen is thus present in the collagen hydrolysate.

In order to enhance the described positive effect, the collagen hydrolysate preferably additionally comprises glycosaminoglycans in an amount by mass of between 20 and 40 percent by weight, particularly preferably between 25 and 35 percent by weight.

Furthermore, advantageously according to the invention, the composition additionally comprises hyaluronic acid. The prominent ability of hyaluronic acid is to bind particularly large amounts of water. Hyaluronic acid is thus essential for water storage in the gelatinous core of the intervertebral discs and for the function thereof as shock absorbers. It has been observed according to the invention that the shock-absorbing function is significantly maintained or enhanced if hyaluronic acid is supplied in combination with N-acetyl-glucosamine as a structural component of hyaluronic acid. Hyaluronic acid supplied directly is quickly available and, in addition, stimulates in a surprisingly pronounced manner the endogenous synthesis of hyaluronic acid by means of supplied N-acetyl-glucosamine.

Advantageously according to the invention, hyaluronic acid is provided in the form of a constituent of a collagen hydrolysate. The collagen hydrolysate preferably comprises hyaluronic acid in an amount by mass of between 5 and 15 percent by weight and particularly preferably between 8 and 12 percent by weight. Such an amount by mass of hyaluronic acid has been found to be particularly effective. In addition, hyaluronic acid is bound in the collagen hydrolysate in such a manner that, on oral ingestion, as large an amount of hyaluronic acid as possible passes through the stomach. Gastric acid present in the stomach would otherwise decompose a large part of the hyaluronic acid. The collagen hydrolysate thus serves, as it were, as a carrying case with which such acid-sensitive hyaluronic acid is carried through the stomach. Only in the largely neutral medium of the duodenum is the collagen hydrolysate enzymatically cleaved. Hyaluronic acid is thereby freed from the collagen hydrolysate and then passes from the small intestine into the bloodstream and from there into the intercellular tissue fluid of the body as a whole.

The collagen hydrolysate further has, in an advantageous manner according to the invention, relative to N-acetyl-glucosamine, a mass ratio of from 2.0 to 4.0, preferably from 2.5 to 3.5 and particularly preferably of 3.0. With such a mass ratio, a particularly good interaction could be achieved in particular in the treatment of back pain.

Furthermore, the composition according to the invention preferably additionally comprises lysine, in particular naturally occurring L-lysine. L-Lysine is an essential amino acid for humans. This means that L-lysine is not formed by the body itself but must be supplied externally.

L-Lysine stimulates the formation and activity of bone-forming cells. In addition, L-lysine serves for muscle growth, promotes the release of growth hormones and stimulates the immune system. Furthermore, L-lysine contributes towards regulating a nitrogen balance in the muscles and promotes calcium storage in the bones, which counteracts the development of osteoporosis by slowing it down. With the supply of L-lysine in addition to N-acetyl-glucosamine, a positive effect on the organism as a whole is achieved, which additionally enhances the positive effect of N-acetyl-glucosamine in particular against vertebral column pain.

A particularly pronounced effect is achieved with a mass ratio, advantageous according to the invention, of lysine relative to N-acetyl-glucosamine of from 0.18 to 0.38, preferably from 0.23 to 0.33, particularly preferably of 0.28.

Furthermore, according to the invention the composition preferably comprises vitamin C. Vitamin C as a collective term includes L-(+)-ascorbic acid and its derivatives having the same action as L-(+)-ascorbic acid. Vitamin C is a coenzyme for the enzyme prolyl-4-hydroxylase, which is required for the biosynthesis of collagen. In addition, vitamin C in combination with L-lysine promotes a covalent crosslinking of adjacent molecules in collagen. Collagen is thus additionally stabilised by vitamin C, which has strengthening effects on the entire connective tissue and thus on connecting structures of the vertebral column in conjunction with the intervertebral discs.

A substantial strengthening effect is achieved with a mass ratio, advantageous according to the invention, of L-(+)-ascorbic acid relative to N-acetyl-glucosamine of from 0.20 to 0.40, preferably from 0.25 to 0.35 and particularly preferably 0.30.

The composition can preferably particularly effectively be taken in conjunction with anti-inflammatory and pain-relieving medicaments which contain, for example, diclofenac, or 2-{2-[(2,6-dichlorophenyl)amino]phenyl}acetic acid, as active ingredient.

Particularly preferably, physiotherapy treatment can additionally be carried out, in particular in respect of an alleviation of back pain. It has thereby been found that the composition according to the invention has an accelerating effect on the success of physiotherapy treatment.

The invention is further directed to a physiologically active composition which comprises at least two components, of which a first component is silica and a second component is N-acetyl-glucosamine or a derivative of N-acetyl-glucosamine. In particular, N-acetyl-glucosamine is present in such a derivative as the basic structure, and a different atom or a different atom group is provided in place of an H atom of the N-acetyl-glucosamine. The term silica includes forms described above as suppliers of silicon as an essential trace element.

Such a composition according to the invention not only has the positive effects already described for the prophylaxis and/or treatment of back pain. It has been shown that the composition according to the invention in a combination of the two components silica and N-acetyl-glucosamine has further mutually enhancing positive effects on the human organism. Silicon is essential for bone development, for the strength and elasticity of cartilage and other connective tissue. Silicon strengthens the skin, hair and nails. In particular, such positive effects are enhanced by means of N-acetyl-glucosamine. The supply of N-acetyl-glucosamine promotes the synthesis of hyaluronic acid as a moisture store of the skin, of connective tissue and of cartilage. In addition, N-acetyl-glucosamine is a structural component for the synthesis of glycosaminoglycans in the connective tissues and an activator for such a synthesis. Crosslinking of glycosaminoglycans is promoted surprisingly strongly by means of silicon. The composition according to the invention thus acts in a synergistically enhanced and increasing manner on the stability and elasticity of all types of connective tissue and in particular also on the skin, hair and nails. The tonicity and thickness thereof could be increased particularly greatly, so that wrinkle formation of the skin could also be counteracted.

In addition, silicon promotes healthy hair growth and shiny hair. Silicon contributes to a hardening of tooth enamel, whereby teeth can be protected from decay. Silicon additionally prevents bleeding of the gums and recession of the gums. Furthermore, silicon is important for a mineralisation process in bones. Silicon in ionised form is one of the most important ions in bone-forming cells and promotes the absorption of calcium from food. Silicon can thus make a significant contribution to preventing the development of osteoporosis. Furthermore, silicon improves the flexibility of blood vessels and can act against arterial diseases. Moreover, silicon has positive effects on the health of the lungs and intestine. In general, silicon reduces vertigo, headache, tinnitus and sleep disorders and stimulates the immune system. Accordingly, many positive secondary effects on the organism as a whole can be achieved with the composition according to the invention in which silica and N-acetyl-glucosamine are combined.

Preferably according to the invention, silica is provided in the form of a component of a bamboo extract. The advantages of the bamboo extract which have already been described apply correspondingly.

For a particularly good interaction, the bamboo extract contains natural silicon dioxide advantageously in the already described amount by mass of from 60 to 90 percent by mass, or percent by weight, preferably from 70 to 80 percent by weight and particularly preferably of 75 percent by weight, based on the bamboo extract. In addition, other substances of natural origin that are valuable for body functions can at the same time be contained in the bamboo extract. Such substances are iron as an essential trace element, calcium as a mineral for bones and teeth, and also choline and betaine. Choline is a vital micronutrient which is converted in the human metabolism to acetylcholine, which serves as a neurotransmitter. Betaine can act prophylactically against arteriosclerosis.

In addition, tabashir as a bamboo extract is known in Tibetan medicine for its stimulating, astringent, antipyretic, strengthening, anticonvulsive and aphrodisiac properties.

Furthermore, the bamboo extract is preferably present in the already described ratio by mass relative to N-acetyl-glucosamine, whereby a particularly good interaction can be achieved.

Furthermore, in an advantageous manner according to the invention, a further component is collagen, which is preferably provided in the form of a constituent of a collagen hydrolysate. In particular, collagen, in addition to the advantages already mentioned, acts to alleviate pain and promote mobility in arthrosis and rheumatic arthritis. Such an action is synergistically enhanced by means of an anti-inflammatory action of N-acetyl-glucosamine on the joints.

In addition, advantageously according to the invention, a further component is hyaluronic acid. Hyaluronic acid has the ability already described to bind particularly large amounts of water. In addition, hyaluronic acid is an important structural component of the connective tissue as well as a main constituent of joint fluid, where it acts as a lubricant for joint movements. Furthermore, in addition to the described advantages, hyaluronic acid also has an anti-inflammatory action in the cartilage tissue. Furthermore, hyaluronic acid promotes a regeneration of the skin and mucosa. Thus, in combination with N-acetyl-glucosamine and silica, the positive effect on the elasticity of cartilage and skin can be significantly enhanced.

Hyaluronic acid is preferably present in the form of a constituent of a collagen hydrolysate in the amount by mass already mentioned.

Collagen hydrolysate and its advantages have already been described, which also applies correspondingly to the composition according to the invention comprising at least two components. The collagen hydrolysate preferably has the ratio by mass relative to N-acetyl-glucosamine already mentioned. A correspondingly good interaction can thus be achieved.

A further component according to the invention is preferably lysine, in particular L-lysine. The advantages already described of an additional combination of L-lysine apply correspondingly, as does the preferred ratio by mass relative to N-acetyl-glucosamine.

In addition, a further component of the composition according to the invention is advantageously vitamin C in preferably the same mass ratio relative to N-acetyl-glucosamine already mentioned. In addition to the described advantages, vitamin C acts as an antioxidant and is necessary for the formation and repair of the connective tissue structures of the skin and cartilage. Such effects synergistically enhance an effect of N-acetyl-glucosamine as an antioxidant and inhibitor of connective tissue degeneration.

Advantageously according to the invention, the composition for use in a method for the prophylaxis and/or treatment of back pain and the composition comprising at least two components are in the form of an oral dosage form. An oral dosage form is easy to handle and is accepted by a user without problems. In addition, the user can easily take the composition himself. It is preferably a solid form of administration, in particular in the form of capsules, tablets, dragées, granules and powders. Addition of the composition to functional foods, such as a bar for healthy nutrition, is also possible. Alternatively or in addition, a liquid dosage form, in particular in the form of drops, stable solutions and/or juices, is also possible. N-Acetyl-glucosamine is distinguished by good water solubility and a sweet taste and is stable in solution over a wide pH range.

When an oral dosage form, in particular in the form of a capsule, is taken, the composition according to the invention passes through the oesophagus into the stomach, where the contents of the capsule are released, in particular by dissolution of a capsule casing of the capsule. Hyaluronic acid initially remains bound in the collagen hydrolysate and is thereby protected against decomposition by means of gastric acid. Furthermore, silica remains bound in the bamboo extract. Only in the duodenum are the bamboo extract and the collagen hydrolysate cleaved enzymatically by means of amylases and peptidases from the pancreas. The collagen hydrolysate, in particular collagen, thus releases hyaluronic acid, and both components are cleaved into parts which can be absorbed by the body. In addition, the bamboo extract releases silica. The ingredients so cleaved are then taken up through the intestinal wall into the bloodstream and pass from there into extracellular tissue of the body as a whole. By means of a special affinity of the intervertebral disc for the cleaved ingredients, in particular as regards pH value and charge, the ingredients accumulate at the intervertebral disc in a particularly high concentration. The ingredients are there quickly incorporated into the intervertebral disc. Collagen thereby serves to synthesise fibrous ring collagen. Silica strengthens and stabilises the bonds, in particular by promoting the crosslinking of collagen. The fibrous ring is thus optimally strengthened. Hyaluronic acid and N-acetyl-glucosamine, which are combined to a complete molecule partly before incorporation into the intervertebral disc and partly in the intervertebral disc, bind an extremely large amount of water. The volume of the gelatinous core is thus increased, which can lead to an increasing distance between the vertebral bodies.

Advantageously according to the invention, the oral dosage form is further adapted to provide N-acetyl-glucosamine in a daily dose of from 100 milligrams to 700 milligrams, preferably from 200 milligrams to 500 milligrams and particularly preferably 300 milligrams. It has been shown according to the invention that N-acetyl-glucosamine achieves the best effect in such a dose. Increasing the dose does not bring about any appreciable increase in the effect. Compared with glucosamine sulfate, which is known as a nutritional supplement, the dose according to the invention is only one third as high.

The invention is additionally directed to a use of the compositions according to the invention as nutritional supplements. The compositions according to the invention can thus be supplied to the body in a simple manner over a longer period. A deficiency of necessary building substances which often exists can be compensated for. Preferably, the intervertebral disc can especially be strengthened in an early stage of wear and thus maintained. To that end, the compositions according to the invention are also suitable for a medicament, a dietetic foodstuff or a medicinal product. As medicaments, the compositions according to the invention have therapeutic uses already described, namely in particular the prophylaxis and/or treatment of back pain, in particular of degenerative vertebral column pain. Preference is given to the treatment of lumbar osteochondrosis.

As dietetic foodstuffs, the compositions according to the invention are a foodstuff for particular medical purposes which serve the specific nutritional requirements of people suffering from specific diseases, disorders or pain. In addition, the dietetic foodstuff is a foodstuff for a specific nutrition, which is processed or formulated in a specific way and is intended for a dietetic treatment of patients.

As a medicinal product, the compositions according to the invention are a substance which is used for medicinally therapeutic purposes. The intended main action thereof is physical or physicochemical.

Accordingly, the invention is also directed to a use of the compositions according to the invention as a medicament, as a dietetic foodstuff or as a medicinal product.

### BRIEF DESCRIPTION OF THE DRAWINGS

An exemplary embodiment of the solution according to the invention will be explained in greater detail hereinbelow with reference to the accompanying drawing, in which:

FIG. 1 is a schematic overview of an exemplary embodiment of a physiologically active composition according to the invention.

FIG. 2 is a schematic overview of a method for the production of the exemplary embodiment according to FIG. 1.

FIG. 3 is a schematic overview of a method for the production of N-acetyl-glucosamine for the exemplary embodiment according to FIG. 1.

### DETAILED DESCRIPTION

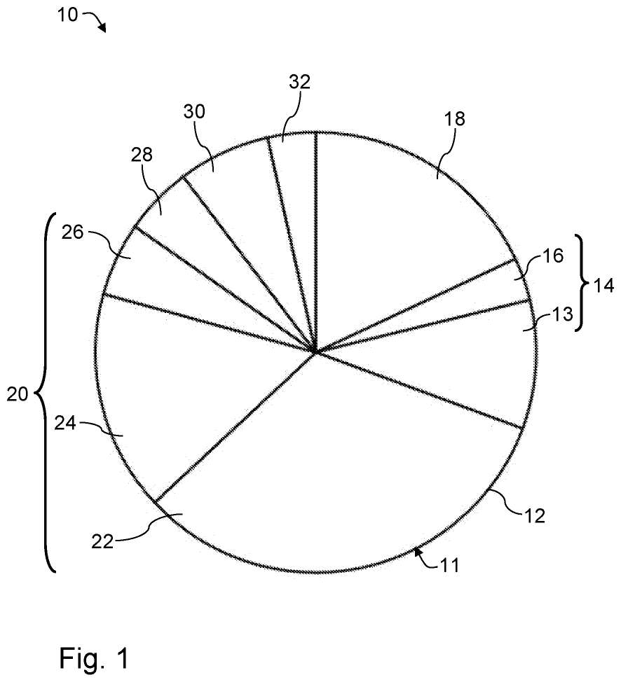

FIG. 1 illustrates a physiologically active composition 10, as is contained in a capsule 11 as an oral dosage form. The capsule 11 serves as a nutritional supplement for maintaining and building up the intervertebral disc.

To that end, the capsule 11 has a transparent and hard capsule casing 12 of hydroxypropylmethylcellulose. Inside the capsule casing 12 is the composition 10 in the form of an active ingredient mixture of several components.

A first component of the composition 10 is silica 13, which is present in the form of natural silicon dioxide in an amount by mass of 75 percent by weight in the form of a constituent of a bamboo extract 14. The bamboo extract 14 is further present in an amount of 70.0 milligrams in the capsule casing, which gives 52.5 milligrams of natural silicon dioxide as the silica 13. A residual amount 16 of the bamboo extract 14, with 17.5 milligrams, amounts to 25 percent by weight, based on the bamboo extract 14, and comprises calcium, choline and betaine.

A second component of the composition 10 is N-acetyl-glucosamine 18, or N-acetyl-D-glucosamine, in an amount of 100.0 milligrams. The bamboo extract 14 is thus present in a mass ratio of 0.7 in relation to N-acetyl-glucosamine 18.

The composition 10 further includes collagen hydrolysate 20 in an amount of 300.0 milligrams. The collagen hydrolysate 20 comprises as a further component collagen 22 in an amount by mass of from 60 to 70 percent by weight, in the present case 60 percent by weight in an amount of 180 milligrams. Collagen 22 is hydrolysed and is preferably in the form of type II collagen.

The collagen hydrolysate 20 additionally comprises as a further component approximately 30 percent by weight mucopolysaccharides or glycosaminoglycans 24 in an amount of approximately 90.0 milligrams. There are further provided in the collagen hydrolysate 20 approximately 10 percent by weight hyaluronic acid 26 in an amount of approximately 30 milligrams as an additional further component of the composition 10.

In total, the collagen hydrolysate 20 has a mass ratio relative to N-acetyl-glucosamine 18 of 3.0.

In addition, lysine 28 is present in a stabilised form as L-lysine hydrochloride in an amount of 35.0 milligrams as a further component of the composition 10. The amount of L-lysine hydrochloride of 35.0 milligrams gives an effective amount of L-lysine of 28.0 milligrams. Lysine 28 thus has a mass ratio of 0.28 relative to N-acetyl-glucosamine 18.

As a further component the composition 10 comprises L-(+)-ascorbic acid, or ascorbic acid, as vitamin C 30 in an amount of 37.5 milligrams. A stability supplement of 25 percent by weight is included therein, in order to compensate for a loss of vitamin C 30 that occurs on prolonged storage. A mass ratio of vitamin C 30 to N-acetyl-glucosamine 18 is approximately between 0.38 and 0.30, depending on the loss on storage.

There are further present in the composition 10 19.5 milligrams of magnesium stearate 32 of vegetable origin as an auxiliary agent.

With 100.0 milligrams of N-acetyl-D-glucosamine 18 of the composition 10 per capsule 11, a particularly effective dose can be achieved with an intake of four capsules 11 per day.

For a particularly good effect, in particular in the case of acute pain, the capsules 11 should be taken in an amount of two times two capsules 11 per day over a period of from two to four weeks. Uptake of the components as active ingredients or nutrients in the body is thus achieved. Thereafter, the intake can be reduced to two capsules 11 per day, divided into two times one capsule 11, for a period of eight weeks. Preference is given to a duration of intake of twelve weeks. The capsules should be taken with copious amounts of liquid after eating.

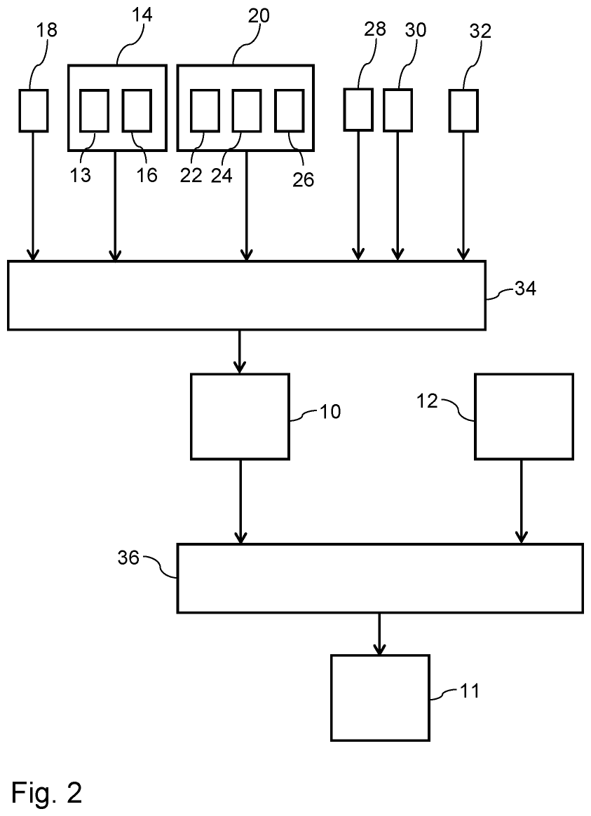

FIG. 2 shows a production method for the capsule 11. The bamboo extract 14, N-acetyl-glucosamine 18, collagen hydrolysate 20, lysine 28 and vitamin C 30 as active ingredient components and magnesium stearate 32 as auxiliary agent are thereby mixed in a step 34 at an ambient temperature of approximately 20° C. Thereafter, in a step 36, encapsulation of the mixed composition 10 so obtained into an empty capsule casing 13 is carried out at 20° C. The capsule 11 is obtained.

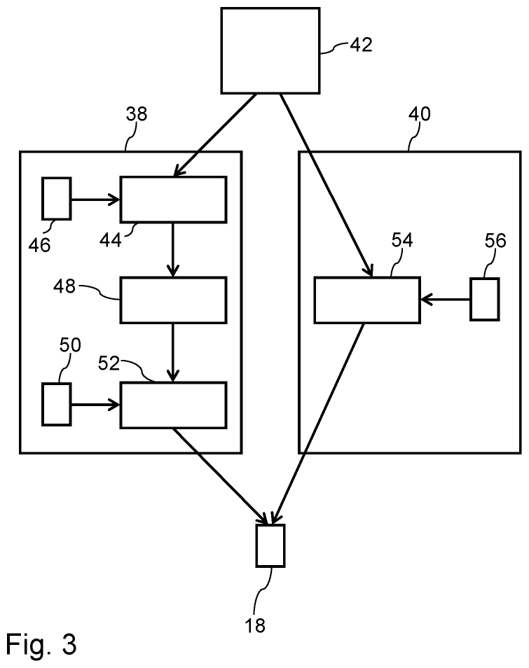

FIG. 3 shows two fundamentally different methods 38 and 40 for producing the active ingredient N-acetyl-D-glucosamine 18. In both cases, natural chitin 42 is used as starting material.

Chitin 42 is a polysaccharide which consists throughout of β-1,4-glycosidically linked N-acetyl-glucosamine molecules. Chitin 42 is thus particularly suitable for obtaining N-acetyl-glucosamine 18. In both methods 38 and 40, chitin 42 from shells of crustaceans is used.

One method 48 is a partially synthetic method, in which natural chitin 42 is subjected to a step 44 of acid hydrolysis, or cleavage. Chitin 42 is thereby cleaved into its individual structural components by means of a strong acid 46. The acid 46 additionally effects cleavage of the acetyl group from the nitrogen of the N-acetyl-glucosamine 18.

In a first variant for the present exemplary embodiment, concentrated hydrochloric acid is used as the acid 46. Glucosamine hydrochloride is thus formed as an intermediate product 48 in the step 44 of acid hydrolysis, and is subsequently concentrated, crystallised and purified. The intermediate product 48 glucosamine hydrochloride so obtained is dissolved in water and reacted with acetic anhydride 50 in a step 52 of re-acetylation to N-acetyl-glucosamine 18. After crystallisation and purification by means of ethanol, N-acetyl-glucosamine 18 is obtained in a purity of 97.5 percent.

In an alternative variant, sulfuric acid is used instead of hydrochloric acid as the strong acid 46 in the method 38, which leads to glucosamine sulfate as the intermediate product 48.

In a further alternative variant, N-acetyl-glucosamine 18 is obtained by means of the other method 40 directly from a cleavage of natural chitin 42. To that end, chitin 42 is swelled with phosphoric acid and then neutralised and subjected in a step 54 to enzymatic hydrolysis by means of an enzyme 56. The enzyme 56 is chitinase. When the hydrolysis is complete, and after subsequent filtration and crystallisation, N-acetyl-glucosamine 18 of completely natural origin is available particularly purely, quickly and simply.

A prospective study which is currently ongoing is described hereinbelow, the interim results of which already demonstrate the activity of the physiologically active composition 10.

In the study, in particular the effects of an administration of the composition 10 to patients with lumbar osteochondrosis, or lumbar vertebral osteochondrosis, are studied.

Such lumbar osteochondrosis is generally observed as a degeneration of the intervertebral disc, which contributes to pain, loss of function of the lower back and absence from work. This problem is to be observed especially in the industrialised world. There is still little understanding of the primary cause of lumbar osteochondrosis. Multi-factorial aspects, such as hereditary, anatomical, traumatic, nutritional, inflammatory and metabolic factors, are probably involved in the progression of the disease.

In addition to a surgical intervention, which is often undesirable, there are various non-surgical therapies for lumbar osteochondrosis, with which pain is to be reduced and normal functions can be regained. Such non-surgical therapies are pharmaceutical pain therapies with, for example, analgesics, non-steroidal anti-inflammatory medicaments, muscle relaxants and/or opiates. Also included are physical therapies, such as, for example, physiotherapy, electrotherapy, ultrasound and/or heat treatment. In addition, treatment with injections is known, such as, for example, trigger point injections and injections to release nerve blocks. Appropriate nutrition alone probably cannot prevent lumbar osteochondrosis, while nutrition management could delay its progression.

In general, nutritional supplements are used for nutrition management and offered anecdotally in clinical practice, despite often contradictory statements and scientific explanations regarding the effectiveness of the individual substances. Moreover, dietetic measures pose a low health risk, so that there are scarcely any studies which have investigated such non-surgical treatment possibilities.

Accordingly, the still ongoing study was begun, which is a prospective, double-blind for patient and investigator, placebo-controlled, 1:1 randomised study of the adjuvant treatment of lumbar osteochondrosis.

The study is to evaluate the effectiveness of the composition 10 as a nutritional supplement in the treatment of lumbar osteochondrosis.

To that end, the composition 10 is used as a nutritional supplement. It bears the trade name vertebene®. The composition 10, with its combination of ingredients, or nutrients, contains substances which occur naturally in the intervertebral disc, such as, for example, collagen fibres and proteoglycans, such as aggrecan. Such a composition 10 should contribute towards improving back pain and/or at least maintaining vertebral column function.

The two primary aims of the study are firstly to evaluate pain and function of the vertebral column and secondly to measure the distance between two vertebral bodies by means of magnetic resonance tomography (MRT), before and after supplementation with the composition 10.

For the study, patients are chosen who go to see an orthopaedic specialist because of lumbar osteochondrosis.

The chosen patients are allocated at random either to a nutritional supplement group, or verum group, or to a placebo group in the ratio 1:1.

The study is a double-blind study, in which neither the patients nor the investigator know who is receiving which test preparation. The test preparations are the composition 10 as the verum, or active substance, and a control preparation as the placebo. Such blinding must not be broken in the course of the ongoing study but only on conclusion of the study, that is to say when the last patient is finished.

Supplementation is carried out for an intervention period of three months. To that end, the patients of both groups take per day two times two capsules of the composition 10 or of the placebo. In addition, the patients are instructed to carry out specific physical exercises.

Data listed hereinbelow are acquired and collected at the beginning of the study, prior to supplementation, as the starting value and after supplementation.

Pain and function are assessed according to subjective patient questionnaires. These include a questionnaire according to the “Oswestry Disability Index” (ODI), in which the patients evaluate the limitations they experience in different functional areas including pain intensity. As further subjective parameters, pain is assessed according to the visual analogue scale (VAS) and quality of life is assessed according to the “short form 12” (SF-12) questionnaire. Questions are asked about dietary habits according to the “food frequency questionnaire” (FFQ) and about physical activity according to the “Global Physical Activity Questionnaire” (GPAQ) of the World Health Organisation (WHO). In addition, a general assessment of the effectiveness of, or satisfaction with, the nutritional supplement is made.

Within the scope of clinical and radiological examinations, the distance between two vertebral bodies, the structure and height of the vertebral bodies and the bone marrow structure are measured by means of magnetic resonance tomography (MRT). In addition, an X-ray examination is carried out in order to determine the distance between two vertebral bodies and to evaluate the osteochondrosis.

A blood sample is additionally taken. Nutritional and inflammation blood parameters and blood parameters relating to oxidative stress are thereby determined. A reverse transcriptase polymerase chain reaction (RT-PCR) for the expression of chondrogenic genes is also carried out.

Since the study is currently not complete, the blinding has consequently also not yet been broken. Despite such a data situation, a current interim state shows the following, promising interim results.

Of the planned 100 patients (50 verum, 50 placebo), 20 patients have so far been recruited. Of these, twelve patients have been included in the study. Ten of these twelve patients have completed the three-month intervention.

Of these ten patients, six patients showed an improvement in symptoms, in two patients the result stayed the same, and in a further two patients there was a slight worsening of the symptoms. The worsening could be due to administration of the placebo.

Analysis of the MRT images also showed very similar results. In 50 percent of the patients, the condition has improved by one degree relative to the starting point. In 30 percent of the patients, the condition has remained the same. In 20 percent, the condition has become worse compared to the starting point, which again could be due to administration of the placebo.

A comparison between verum and placebo is not yet possible because the blinding has not been broken. However, these interim results already give very clear evidence that, by means of an intake of the composition 10, an improvement in symptoms can be achieved at least in the case of lumbar osteochondrosis.

The composition 10, in particular after completion of the study and further positive results, can be offered as an adjuvant therapy for the prophylaxis and/or treatment of back pain, in particular in the case of degenerative vertebral column pain, or intervertebral disc pain, such as lumbar osteochondrosis.

Finally, it should be noted that all the features which are mentioned in the application documents and in particular in the dependent claims are to be accorded independent protection, also individually or in any desired combination, despite the formal dependence on one or more specific claims which has been made.

### LIST OF REFERENCE NUMERALS

10 composition
11 capsule
12 capsule casing
13 silica
14 bamboo extract
16 residual portion bamboo extract
18 N-acetyl-glucosamine
20 collagen hydrolysate
22 collagen
24 mucopolysaccharides or glycosaminoglycans
26 hyaluronic acid
28 lysine
30 vitamin C
32 magnesium stearate
34 mixing step
36 encapsulation step
38 method for producing N-acetyl-glucosamine
40 method for producing N-acetyl-glucosamine
42 chitin
44 acid hydrolysis step
46 strong acid
48 intermediate product
50 acetic anhydride
52 re-acetylation step
54 enzymatic hydrolysis step
56 enzyme

1-10. (canceled)

11. A physiologically active composition comprising N-acetyl-glucosamine or a derivative of N-acetyl-glucosamine for use in a method for the prophylaxis and/or treatment of back pain.

12. The composition according to claim 11, further comprising silica.

13. The composition according to claim 12, wherein the silica is provided in the form of a constituent of a bamboo extract.

14. The composition according to claim 11, further comprising collagen.

15. The composition according to claim 14, wherein the collagen is provided in the form of a constituent of a collagen hydrolysate.

16. The composition according to claim 11, further comprising hyaluronic acid.

17. The composition according to claim 16, wherein the hyaluronic acid is provided in the form of a constituent of a collagen hydrolysate.

18. The composition according to claim 11, further comprising at least one of lysine and vitamin C.

19. A physiologically active composition which comprises at least two components, of which a first component is silica and a second component is N-acetyl-glucosamine or a derivative of N-acetyl-glucosamine.

20. The composition according to claim 19, wherein silica is provided in the form of a constituent of a bamboo extract.

21. The composition according to claim 19, further comprising at least one of collagen, hyaluronic acid, lysine and vitamin C.

22. The composition according to claim 11 in the form of an oral dosage form.

23. The composition according to claim 19 in the form of an oral dosage form.

24. A nutritional supplement comprising the composition as in claim 11.

25. A nutritional supplement comprising the composition as in claim 19.

26. A method for the prophylaxis and/or treatment of back pain, comprising:
administering a physiologically active composition comprising N-acetyl-glucosamine or a derivative of N-acetyl-glucosamine.
